(19) United States
(12) Patent Application Publication
(10) Pub. No.: US 2023/0027322 A1
(43) Pub. Date: Jan. 26, 2023

(54) THERAPEUTIC MUSIC AND MEDIA PROCESSING SYSTEM

(71) Applicant: Coro Health, LLC, Austin, TX (US)

(72) Inventors: David Schofman, Austin, TX (US); Brian Anderson, Austin, TX (US); Stephen Balkum, Thrall, TX (US)

(73) Assignee: Coro Health, LLC, Austin, TX (US)

(21) Appl. No.: 17/836,767

(22) Filed: Jun. 9, 2022

Related U.S. Application Data
(60) Provisional application No. 63/208,922, filed on Jun. 9, 2021.

(51) Int. Cl. G06F 16/638; G10H 1/00; G16H 20/70; G06F 16/65; G06F 16/683

(52) U.S. Cl. CPC G06F 16/639; G10H 1/0008; G16H 20/70; G06F 16/65; G06F 16/683; G10H 2210/076; G10H 2240/131

(57) ABSTRACT

Systems, methods, architectures, mechanisms and apparatus for generating an audio segment playlist configured to provoke a physiological response in a listener in accordance with a desired outcome category, comprising: selecting, from a features database, a plurality of audio segments having features associated with both listener information and the desired outcome category; and ordering within the playlist at least a portion of the selected audio segments in accordance with at least one feature progression associated with the outcome category.

### CROSS-REFERENCE TO RELATED APPLICATION

This application claims the benefit of and priority to prior filed co-pending Provisional Application Ser. No. 63/208,922 filed Jun. 9, 2021, entitled THERAPEUTIC MUSIC AND MEDIA PROCESSING SYSTEM (Attorney Docket No. CORO-003), which provisional patent application is incorporated herein by reference in its entirety.

### FIELD OF THE DISCLOSURE

The present disclosure generally relates to therapeutic content in general and, more particularly, to systems adapted to automatically characterizing music/media and constructing therapeutic music/media deliverables configured to affect patient outcome.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Music therapy is presently practice by skilled professionals with individual patients. The delivery of therapeutic music is inherently limited by the number of skilled practitioners available. Thus, delivery of such therapy within institutional environments in inherently impractical and the specific implementation problems associated with such institutional environments have not been addressed.

### SUMMARY

Various deficiencies in the prior art are addressed by systems, methods, architectures, mechanisms and apparatus for generating an audio segment playlist configured to provoke a physiological response in a listener in accordance with a desired outcome category, comprising: selecting, from a features database, a plurality of audio segments having features associated with both listener information and the desired outcome category; and ordering within the playlist at least a portion of the selected audio segments in accordance with at least one feature progression associated with the outcome category. In response to a change in desired playlist runtime, or a change in listener information, further selected audio segments may be added to the playlist or existing audio segments may be deleted from the playlist. The feature progression may be formed using one or more of the following tempo, brightness (timbre), brightness variability, pulse strength, pulse variability, proportion in major key(s), tonal clarity, tonal clarity variability, tonal entropy, or other objectively determinable features. The feature progression may be formed using one or more of acousticness, danceability, energy, intrumentalness, loudness, speechiness, tempo and valence, or other subjective features compound features (i.e., a combination of progressions such as slopes or shapes defining existing features over the playlist). The desired outcome category of the playlist may comprise an energy category, and the feature progression comprises a positive Tempo Feature Slope (TFS). The desired outcome category of the playlist may comprise a relax category, and the feature progression comprises a negative Tempo Feature Slope (TFS).

In some embodiments, prior to inclusion in the feature database, each audio segment is processed using at least two feature extraction tools configured to extract therefrom respective sets of audio segment features. In some embodiments, responsive to common audio segment features extracted by different feature extraction tools being different, an average of the common audio segment features will be included in the feature database. In some embodiments, responsive to a statistical distance between common audio segment features extracted by different feature extraction tools being indicative of a feature extraction error, the feature data indicated as erroneous will not be included in the feature database.

Additional objects, advantages, and novel features of the invention will be set forth in part in the description which follows, and will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

The teachings of the present invention can be readily understood by considering the following detailed description in conjunction with the accompanying drawings, in which:

FIG. 1 depicts a functional block diagram of a method according to one embodiment;

FIG. 2 depicts an exemplary Scheduler administrative user interface according to one embodiment;

FIG. 3 depicts an exemplary Scheduler administrative user interface according to one embodiment;

FIG. 4 depicts a functional representation of a therapeutic music delivery system according to one embodiment;

FIG. 5 depicts a high-level block diagram of a general-purpose computer suitable for use in performing the functions described herein;

FIG. 6 depicts a high-level block diagram of a system according to one embodiment;

FIG. 7 depicts a flow diagram of a method according to one embodiment;

FIG. 8 graphically depicts a process flow in accordance with various embodiments;

FIG. 9 graphically depicts a Tempo Feature Slope (TFS) of an exemplary music prescription generated in accordance with an embodiment;

FIG. 10 graphically depicts a Tempo Feature Slope (TFS) for each of a plurality of energy playlists, along with an average of the TFSs of the plurality of playlists;

FIG. 11 is a histographic representation of the distribution of tempo feature slopes for the energy playlist of FIG. 10;

FIG. 12 depicts a flow diagram of a features database update method according to an embodiment. Specifically, the steps of FIG. 12 are discussed above and herein; and

FIG. 13 depicts a flow diagram of a playlist generation method according to an embodiment.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for pedagogical purposes to aid the reader in understanding the principles of the invention and the concepts contributed by the inventor(s) to furthering the art, and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Some embodiments will be described within the context of a system for providing therapeutic content such as music to residents or patients within the context of an assisted living/managed care environment, hospital or other institution (medical or nonmedical). However, those skilled in the art and informed by the teachings herein will realize that the invention is also applicable to other technical areas and/or embodiments. For example, the invention has applicability within the context of schools, prisons, hospitals and other (typically) institutional settings where music or content based therapy can be delivered to patients.

The terms “patient” and “resident,” which will be used frequently within the context of the below description, are to be broadly construed as referring to any of a patient, student, prisoner, resident and the like associated with an institution. Generally speaking, a resident, patient, student and so on is simply one to whom therapy is delivered.

The terms “music,” “content,” “media” and the like will be used frequently within the context of the below description to describe specific therapies delivered to a patient. These terms are to be broadly construed as substantially interchangeable in terms of a delivered therapy, except where specifically defined as being different.

One embodiment comprises a specific configuration of hardware with four components to create a dynamic and scalable method for delivering custom, individualized therapeutic music to patients. The four components are (1) Scheduler; (2) Player; (3) Administrative User Interface; and (4) Music Prescription Algorithm. Each of these will be discussed briefly below and then in more detail.

Scheduler

Patients in most medical facilities follow a very specific schedule/routine called an ADL (Activities of Daily Living). The Scheduler software specifically maps prescribed musical or other media/content playlists to each patient's ADL's. An example of a schedule for a patient X may comprise: (a) Wake—6 am; (b) Breakfast—8 am; (c) Activity—10 am; (d) Lunch—12 pm; (e) Nap—1 pm; (f) Activity—3 pm; (g) Dinner—5 pm; (h) Free Time—7 pm; and (i) Sleep—9 pm.

The scheduler software plays specific playlists prior to and/or during these activities or transitions between these activities for patients to prepare for these different daily events. Thus, for example, at 5:45 am wake music begins to help a patient slowly and comfortably transition from sleep to wake (and so on). The software may be provided as a set of predetermined templates based on the facility's standard ADLs. The software may also provide staff with the ability to modify each individual's schedule as needed. Changes can be made temporarily (e.g., “just for today”) or permanently.

Player

A wired or wireless media player operates with, illustratively, a laptop or central facility server that can decode and stream multiple music playlists simultaneously. Wired embodiments and other embodiments may also be used. In one embodiment, content is moved to facility servers from external (remote) servers, and then moved to patient computers or presentation devices.

Administrative User Interface

The Administrative User Interface is used by facility administrators and staff to create and edit resident (patient) profiles, playlists, quick play (“on-demand”) parameters and other schedules functions. Administrators such as managers, activities directors, and music therapists have the ability to add resident (patient) profiles, create facility wide schedule templates, assign playlists and customize recurring schedules for residents. Other staff members are able to view patient playlists and create non-recurring adjustments to patient schedules.

M3 Algorithm—Music Prescription Algorithm

The Music Intelligence of the M3 algorithm takes multiple inputs into consideration to determine the appropriate content to be consumed by the patient and the time/circumstances of such consumption. The M3 Algorithm utilizes the various inputs to compile data and create a final product; namely, a Music Prescription comprising a series of custom playlists adapted to provide or encourage a desired patient response. The multiple inputs comprise one or more of:
Patient Assessment—Patient history, background, issues, desired outcome
Musical Assessment—Series of short music clips administered to patient
Music Database—Based on inputs from previous pieces, provides recommendations of “like” music based on the musical characteristics such as (beats per minute, vocal, genre, instrumental, tempo)
Patient Vital Signs—Monitored/recorded throughout assessment
Licensed Music Therapist—Reviews data, provides input.

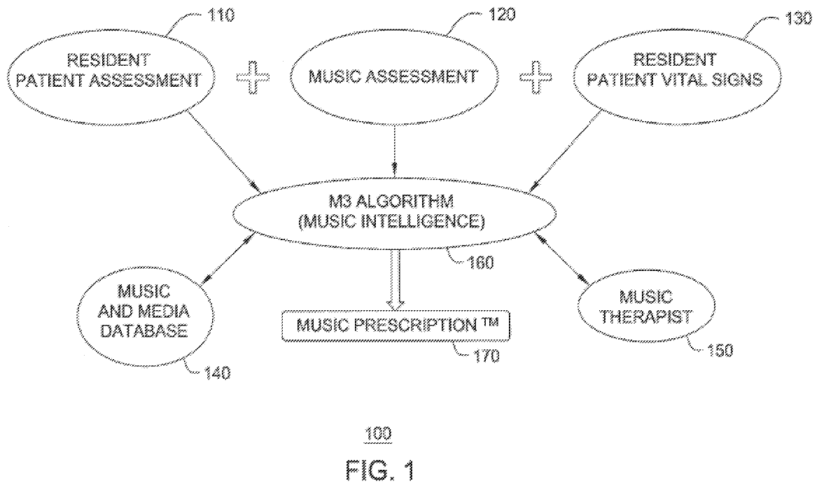

FIG. 1 depicts a functional block diagram a method according to one embodiment. Specifically, FIG. 1 depicts a flow diagram of a method for generating a music prescription. Specifically, the method 100 utilizes a patient assessment 110, music assessments 120, patient vital signs 130 and other information (optional—not shown) associated with a particular patient are provided to an M3 software core algorithm or music intelligence engine 160.

The music intelligence engine 160 uses the provided information to generate a music prescription 170 for the particular patient. The music intelligence engine 160 cooperates with a music and media database 140 to select music or other content appropriate for the particular patient in accordance with the music prescription 170. Optionally, input to the music intelligence engine 160 may also be provided by a music therapist 150.

The music prescription 170 comprises a playlist of specific content such as musical titles appropriate to the particular patient based upon the type of music that the patient enjoys, the type of activity or time of day that the music will be presented to the patient, and the present status (e.g., vital signs) associated with the patient.

The Music Prescription may be considered to be a building of a series of custom, individualized content or music playlists for a patient experiencing a wide range of health issues, such as depression, sleep disorders, pain management, dementia and so on. Helping patients with specific medical issues through the use of content such as music is referred to as the “non-music outcome” to be attained.

Patient vital signs may include any or all of heart rate, respiration rate, body temperature, skin temperature, measurements related to restlessness, measurements related to sleep quality, measurements related to attention level, measurements related to concentration level and so on. Vital signs can also include a smile, a tap of a foot or hand, a change in breathing pattern, change in eye contact and the like. Generally speaking, any time of measurement or quantifiable data associated with a patient may be considered a patient “vital sign” useful in assessing the patient and/or modifying a therapeutic content/music treatment.

In one embodiment, music or content therapy delivered to a patient and intended to promote a restful state (e.g., sleep, relaxation, reduction in agitation, etc.) is modified in response to achieving that state, as indicated by changes in heart rate, respiration rate or other appropriate vital sign (e.g., slowed heart rate, slowed/deep/even breathing).

In one embodiment, music or content therapy delivered to a patient and intended to promote a wake state (e.g., waking up, getting ready for activity or exercise and so on) is modified in response to achieving that state, as indicated by changes in heart rate, respiration rate or other appropriate vital sign (e.g., increased heart rate, quickened respiration rate and so on).

Exemplary components related to patient or consumer assessment 110 and music assessment 120 will now be described along with an exemplary music prescription and distribution method.

Consumer Assessment Component.

A questionnaire and/or interview is given to the consumer, caregiver and/or family. The following types of information may be collected:
Consumer medical history; such as hearing ability, mental cognition, cancer, heart attack, etc.
Consumer background; such as where the patient grew up, traumatic events, gender, race, age, etc.
Consumer current issues; such as Pain, insomnia, stress, depression, etc.
Consumer desired outcome; such as Pain reduction, better sleep, reduce anxiety/depression, gait training, etc.
Consumer schedule: Gathers information to determine the correct time of day for certainly playlists or songs. Examples include ADL (Activities of Daily Living) or current daily routine.

Music Assessment Component

A. Music Questionnaire

A music questionnaire is given to the consumer, care giver, family member, teacher and the like to better understand the specific music preferences.

Examples: Music ability, favorite music, any music make you sad/happy, music dislikes (Live or recorded samples may be played during this questionnaire), favorite color, etc.

B. Music Clips

Series of short music clips administered to consumer. Each clip has certain characteristics.

Purpose: Administrator observes consumer during each clip and (if possible) asks consumer if they like or dislike. Administrator uses data to help in playlist building process. Administrator may be any system programmed to perform these tasks, or personnel appropriate to perform these tasks.

Example: 30 clips of music, 30 seconds in length. (number of clips and length varies based on consumer cognition, ability, responsiveness, etc.)

Vital Signs

Vital signs are monitored while playing the music clips.

Purpose: Vital signs provide a concrete (non-subjective) method in which to understand a consumer's reaction to music. This way, more reliable, more consistent data is gathered.

Examples: Heart rate, blood pressure, pulse ox, respiratory rate, biofeedback, EEG

Vital signs are optionally monitored through a handheld device, while listening to music clips (on same or different device) to determine Music Prescription™

Music Prescription—Playlist/Song Design

Once a specific non-music outcome is identified, information gathered from the Consumer Assessment and Music Assessment are incorporated into creating the Music Prescription™ for the consumer.

The music is then positioned in a specific sequence.

The playlist is then mapped to the participant's routine for the desired outcome at the correct time.

The playlists are for a specific duration and are not played continuously throughout day. No effort by the participant is required to stop the music. Once the sequence has finished, the music stops until the next scheduled time triggers the next music playlist.

In an effort to assist the song selection, a music database is kept. The music database consists of a library of music which is used to pull individual songs together for consumers. Each song has certain characteristics (BPM, tempo, vocal, instrumental, etc.) that are assigned within the database so that they can be grouped.

Once a specific song or playlists of songs is constructed for an individual, those songs or playlists are stored in the database or delivered to the consumer as determined by their Music Prescription™

In various embodiments the methodologies described herein determine music that is by some measure “best” or “positive” for an individual or patient such that a desired therapeutic or behavioral result is obtained. Various embodiments also determine which music has a negative impact on the individual or patient. This music may have a dramatic negative influence on the mood and/or behavior of an individual and, as such, should be avoided (along with music the individual simply does not prefer).

Distribution Method

Software: M3 Scheduler

Scheduler software distributes music to each participant's ADL's or daily routine.

Scheduler has ability to be modified (times of day, music, volume) by the individual or other 3rd party involved in the process or repeat the same song or playlists at the same time each day.

Hardware:

Delivers the Music to the Individual

Individual music can be delivered via wired or wireless delivery to a large population of individuals in a specific setting or independently/directly through a single music playing device (e.g., to an MP3 player).

Individuals can receive the music through headphones (wired or wireless), traditional speakers, speaker pillows, Bluetooth device, hearing aid or other music speaker delivery system.

MP3 Media Server has ability to play multiple playlists to a large population within a specific environment (hospital, nursing home, school, day care, NICU, prison, spa, hotel) or be loaded on a single device (MP3 player) to be used individually by a consumer in their private setting (home, office, airport, car).

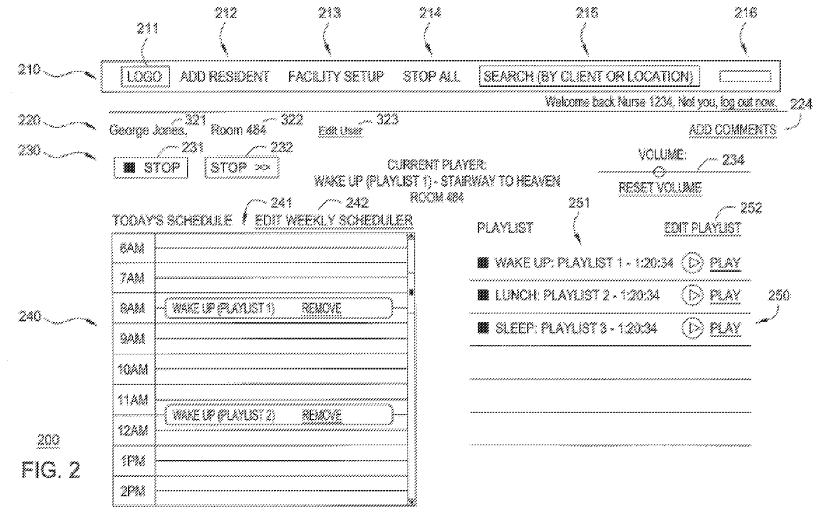

FIG. 2 depicts an exemplary Scheduler administrative user interface according to one embodiment. Specifically, FIG. 2 depicts a user interface display 200 suitable for administrative interaction with the scheduler program to define a daily schedule for particular patient.

The user interface display 200 comprises a header region 210, a patient identification region 220, a current content control region 230, a daily schedule region 240 and a playlist region 250.

The header region 210 is depicted as including a logo 211 (e.g., the logo of the hospital or institution), an “Add Resident/Patient” button 212, a “Facility Set up” button 213, a “Stop All” button 214, a search input box 215, and a “search” button 216.

Selecting the “Add Resident/Patient” button 212 invokes a user interface screen that enables an administrator to enter details associated with a new patient or resident at the facility. Selecting the “Facility Set up” button 213 invokes a user interface screen that enables an administrator to enter details associated with the facility set up, such as changes to the details of the computer or communications equipment supporting the system. Selecting the “Stop All” button 214 invokes a cessation of content presentation to the patient. Entering a search term into the search input box 215 and selecting the “search” button 216 invokes a user interface screen that enables an administrator to retrieve details regarding the patient, client, location, facility and so on.

The patient identification region 220 is depicted including a patient's main display 221, patient room display 222, “edit user” button 223 and “add comments” button 224.

The current content control region 230 is depicted as including a “stop” playing content button 231, a “skip to next” content in list button 232, a “currently playing” content identifier 233 and a volume control slider 234.

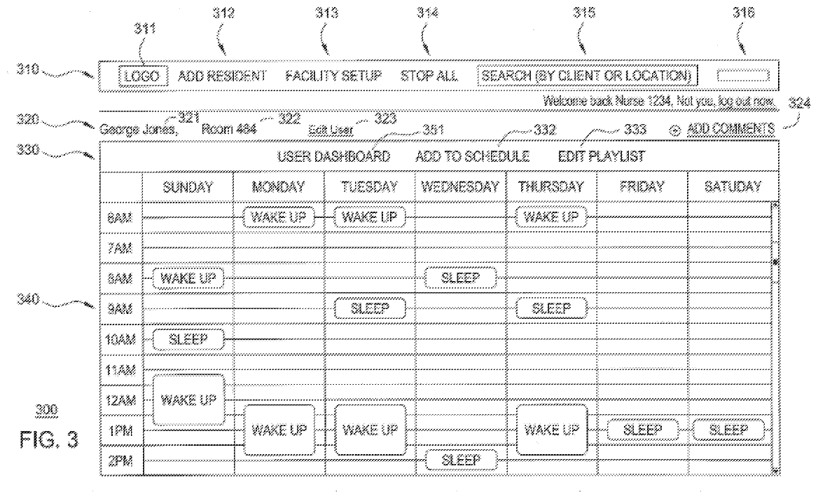

The daily schedule region 240 is depicted as including a graphical representation of the patient's schedule including content presentation times 241 as well as a “edit weekly scheduler” button 242. Selecting the “edit weekly scheduler” button 242 invokes a user interface screen that enables an administrator to enter details pertaining to the weekly schedule associated with the patient. This user interface screen will be discussed in more detail below with respect to FIG. 3.

The playlist region 250 is depicted as displaying a daily content playlist 251 and an “edit playlist” button 252. Selecting the “edit playlist” button 252 invokes a user interface screen that enables an administrator to modify the daily playlist.

FIG. 3 depicts an exemplary Scheduler administrative user interface according to one embodiment. Specifically, FIG. 3 depicts a user interface display 300 suitable for administrative interaction with the scheduler program to define a weekly schedule for particular patient.

The user interface display 300 comprises a header region 310, a patient identification region 320, a context control region 330 and a weekly schedule region 340.

The header region 310 and patient identification region 320 include respective sub element that operate in substantially the same manner as those described above with respect to header region 210 and patient identification region 220. As such, the description of these regions and their sub elements will not be repeated.

The context control region 330 is depicted as displaying a “user dashboard” button 331, an “add to schedule” button 332 and an “edit playlist” button 333. Selecting the “user dashboard” button 332 invokes a user interface screen that enables an administrator to modify various system-level parameters. Selecting the “add to schedule” button 332 invokes a user interface screen that enables an administrator to add content/playlist items as well as other items to the patient's schedule. Such otherwise may comprise, illustratively, scheduled medical exams, transport to other facilities, doctor visits, family visits and so on. Selecting the “edit playlist” button 333 invokes a user interface screen that enables an administrator to edit the content playlist associated with the patient.

The weekly schedule region 340 is depicted as a graphical representation of a patient's weekly schedule, illustratively a grid comprising time as a function of day of the week, where scheduled items are displayed therein.

The administrative user interface screens depicted above with respect to FIGS. 2-3 may comprise Web applications invoked within a browser program running on an administrative computer. The administrative computer may be local with respect to the facility or remote with respect to the facility (for example, at an administrator's house). In one embodiment, the Administrative User Interface application is a web application written in C#. This permits rapid development, rich automated testing, and easy remote access for users and other support personnel.

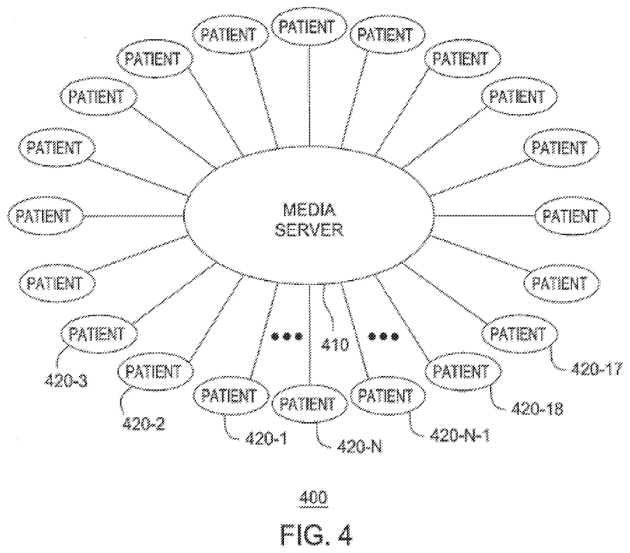

FIG. 4 depicts a functional representation of a therapeutic music delivery system according to one embodiment. The system 400 FIG. 4 comprises a media server 410 in communication with a plurality of patient processing/presentation devices denoted as patient devices 420-1, 420-2 and so on through 420-N.

Each of the patient devices 420 comprises, illustratively, a computing device communicating with the media server 410 and with a presentation device (not shown), such as an audio presentation device (e.g., speakers or earphones) or an audiovisual presentation device (e.g., a television or other display device). Each patient devices 420 received content, commands and/or other data from the media server 410 and responsively present the received content to the patient at the scheduled time.

In one embodiment, communication between the media server for 10 and patient devices 420 is provided via an Ethernet or other hardwired network connection. In other embodiments, such communication is provided via a wireless network, such as an 802.11, WiMax or GPRS network. The media server for 10 and patient devices 420 include appropriate networking functionality to achieve the desired interconnectivity.

In one embodiment, communication between the various functional modules implementing systems according to the present embodiments are handled via a service bus architecture. This bus architecture provides significant separation of concerns or effort for developers, which in turn speeds development and ensures rigorous programming practices. More importantly, the loose coupling of the modules afforded by the bus architecture enables scalability and flexible deployment of processing power. This allows the deployment footprint to scale from a single, self-contained server for the smallest facilities to the largest facilities where a player-server per floor or wing is required. Thus, in various embodiments, both single and multiple processing elements are envisioned to support application processing loads and/or other processing loads.

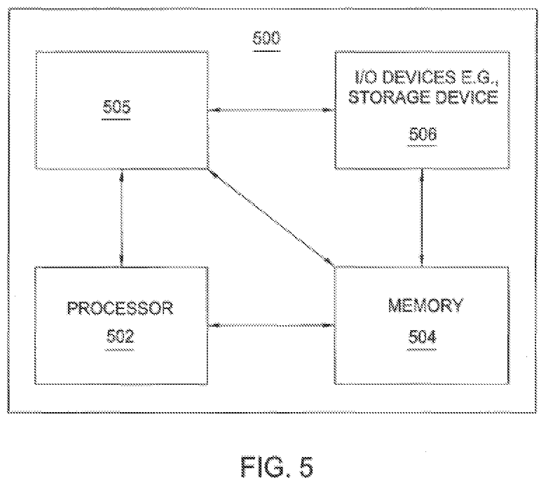

FIG. 5 depicts a high-level block diagram of a general-purpose computer suitable for use in performing the functions described herein. Specifically, FIG. 5 depicts a high-level block diagram of a general-purpose computer suitable for use in performing the functions described herein. As depicted in FIG. 5, system 500 comprises a processor element 502 (e.g., a CPU), a memory 504, e.g., random access memory (RAM) and/or read only memory (ROM), an RMT management module 505, and various input/output devices 506 (e.g., storage devices, including but not limited to, a tape drive, a floppy drive, a hard disk drive or a compact disk drive, a receiver, a transmitter, a speaker, a display, an output port, and a user input device (such as a keyboard, a keypad, a mouse, and the like)).

It should be noted that the present invention may be implemented in software and/or in a combination of software and hardware, e.g., using application specific integrated circuits (ASIC), a general purpose computer or any other hardware equivalents. In one embodiment, the various processes can be loaded into memory 504 and executed by processor 502 to implement the functions as discussed above. As such the processes (including associated data structures) of the present invention can be stored on a computer readable medium or carrier, e.g., RAM memory, magnetic or optical drive or diskette, and the like.

It is contemplated that some of the steps discussed herein as software methods may be implemented within hardware, for example, as circuitry that cooperates with the processor to perform various method steps. Portions of the functions/elements described herein may be implemented as a computer program product wherein computer instructions, when processed by a computer, adapt the operation of the computer such that the methods and/or techniques described herein are invoked or otherwise provided. Instructions for invoking the inventive methods may be stored in fixed or removable media, transmitted via a data stream in a broadcast or other signal bearing medium, and/or stored within a memory within a computing device operating according to the instructions.

The embodiments described herein generally provide customized or individualized content such as video or music and, more generally, integrate a specific process, technology and human interaction (music therapist, musician, etc.) to determine an optimal song selection and sequence for a specific non-musical outcome. The process is comprised of a consumer assessment, music assessment, music prescription™ (playlist) and distribution method. These playlists are then mapped to one's schedule or ADL's (Activities of Daily Living) for maximum benefit. Proprietary software has been developed to offer this solution as well as a unique combination of hardware components.

Various embodiments of the technology utilize off the shelf components that are easy to setup and maintain, as well as development and support resources that are readily available. A platform supporting one embodiment comprises a .Net service where the software is coded using the C-sharp (C#) programming language within the context of a Windows operating system. Developers using this platform and others like it are able to create a robust system including significant automated testing to ensure a long life for the source code and the product.

In one software embodiment, there are three primary modules: the Scheduler, the Player, and the Administrative User Interface. The Scheduler is the heart of the system. The Scheduler monitors the data repository for all patient schedules and playlists. It triggers the player to deliver specific playlists for individual patients at designated locations. The Scheduler is, illustratively, invoked as a background process written in C# or other programming language. The Player is responsible for decoding and streaming audio from multiple sources and delivering the streams to unique destinations. Similar to the Scheduler, the Player may optionally run as a background process. To achieve the required performance level, in one embodiment the Player is written in C#, but with significant optimizations in C++.

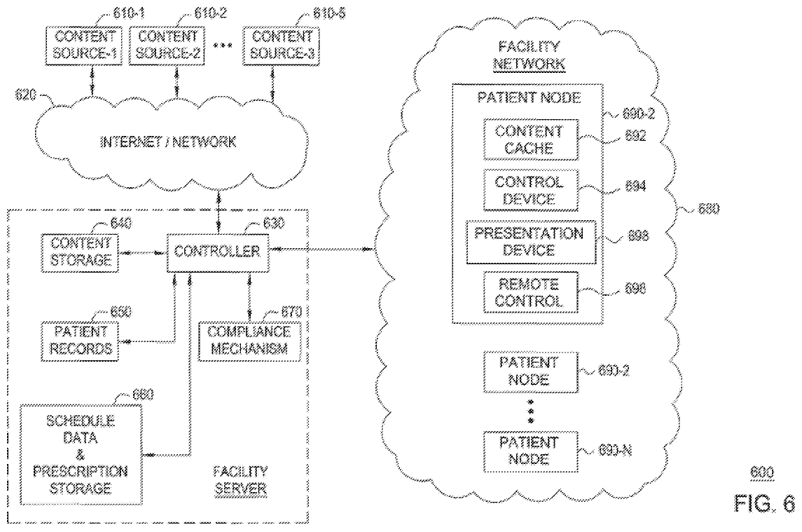

FIG. 6 depicts a high-level block diagram of a system according to one embodiment. Specifically, the system 600 of FIG. 6 is adapted to package content such as music or video programming for scheduled delivery to patients within the context of an extended care center, nursing home or other facility providing content/music therapy to its patients.

The system 600 FIG. 6 comprises, illustratively, equipment remote from the facility such as content sources, as well as equipment local to the facility such as administrative equipment and patient equipment. Some of the local equipment may also be remotely located with respect to the facility, as will be described in more detail below.

Generally speaking, content may be provided initially from any source such as a remote content provider, optical and magnetic media and so on. The content is delivered to patients via patient network nodes having content presentation capabilities were associated with a content presentation device. The specific content delivered to patients, and is scheduled for delivery of such content, is described in more herein with respect to the various figures.

Referring to FIG. 6, remote equipment comprising a plurality of content sources denoted 610-1, 610-2 and so on through 610-N (collectively content sources 610) communicate content to the facility via a network such as the Internet 620. The content sources 610 are depicted as being remote with respect to a facility including administrative equipment. However, in various embodiments some or all the content sources 610 may be local with respect to a facility. The content sources 610 are also depicted as comprising a plurality of content sources. However, in various embodiments a single content sources 610 is used to provide content to the facility.

Local equipment within the facility includes administrative equipment comprising controller 630, a content storage device 640, a patient records database 650, a schedule data and prescription storage database 660 and a facility network 680. Generally speaking, administrative equipment comprises one or more computers, data servers, storage devices, communications devices and the like adapted to perform the methodologies described herein.

Local equipment within the facility also includes patient equipment comprising patient network nodes 690-1, 690-2 and so on through 690-N (collectively patient network nodes 690). Generally speaking, patient equipment comprises a computer or computing device including a communications capability and a content storage capability for receiving and providing content to a patient accessible presentation device.

The controller 630 comprises a single or multiple server control computer including processor, memory and input output (I/O) functionality suitable for performing the various functions described herein. In particular, the controller 630 communicates with the one or more content sources 610 via the Internet 620 to receive content such as music, video programming, electronic mail, voicemail, messages and the like.

The content storage device 640 comprises a mass storage device or content server for storing content. The content storage device 640 communicates with the controller 630 and is operative to store content received via the controller 630 or via some other mechanism (not shown). The content storage device 640 also provides stored content to the controller 630 and/or as directed by the controller 630. Provide content may be streamed to/through the controller 630 The content storage device 640 may be implemented using magnetic or optical media, a redundant array of inexpensive devices (RAID), or other local mass storage mechanism. Alternatively, the content storage device 640 may be located remotely from the facility and accessed via the Internet 620, facility network 680 or some other communication means (not shown).

The patient records database 650 comprises a secure database for storing patient medical information in conformance with the various federal, state and local requirements pertaining to medical privacy. The controller 630 interacts with the patient records database 650 to retrieve patient information as necessary, such as to implement the algorithms discussed herein. The controller 630 also provides updated patient information to the patient records database 650, such as changes in prescription (pharmaceutical, content, music or otherwise), position reports, administrator notes pertaining to patient function or therapeutic response, updates to medical conditions and the like.

The schedule data and prescription storage database 660 comprises a secure database for storing patient schedule information including scheduled content/music prescription information. The patient schedule information is generated by administrative personnel using the various interfaces/algorithms discussed above a check to the various figures.

The optional compliance mechanism 670 comprises a mechanism to ensure that facility procedures to inadvertently fall out of regulatory compliance or compliance with facility procedures. Examples of such compliance include medical prescription contraindication crosschecking, content/music therapy compliance with evolving patient requirements and so on.

The facility network 680 comprises a wired or wireless network for conveying content/music, data and other information between administrative equipment and patient equipment. A wired network may comprise a dedicated Ethernet network, a power line network or other wired mechanism for conveying network traffic. A wireless network may comprise an 802.11 type network, a WiMax network, a general packet radio service (GPRS) network or other wireless mechanism for conveying network traffic. Generally speaking, network traffic conveyed to patient equipment or between administrative and patient equipment may comprise any electrical, optical or radio broadcast technology network.

It is contemplated that the patient equipment receives and presents content according to the schedule associated with individual patients. It is also contemplated in various embodiments that patient monitoring data is conveyed from the patient equipment to administrative equipment for subsequent processing (e.g., outcome tracking, dosage monitoring, alarm indication and so on).

Patient network nodes 690 comprise a content cache 692 as well as a control device 694 adapted to convey content/music to an appropriate presentation device 696. Optionally, patient network nodes 690 for the comprise the remote control device 698, which device may be used to control the presentation device 696 and, optionally, it interact with a respective control device 694 and/or the administrative equipment controller 630.

In one embodiment, the content/music is streamed to the presentation device 696 via a session established between the control device 694 and the controller 630. In other embodiments, the content/music for each patient is stored in the content cached 692 a the patient notes 690 associate with the patient. As previously noted, the presentation device 696 may comprise any device suitable for presenting audio or audiovisual content to a patient.

In one embodiment, the presentation device 696 also includes massage equipment and/or equipment for imparting tactile stimuli to a patient. In this embodiment, imparted tactile stimuli may be synchronized with a presentation of content/music.

In one embodiment, the presentation device 696 also includes aromatherapy equipment for imparting aroma stimuli to a patient. In this embodiment, imparted aroma stimuli may be synchronized with a presentation of content/music and/or any tactile stimuli.

In one embodiment, patient equipment is implemented via a plug computer which includes a wireless network interface adapted to communicate with administrative equipment. In one embodiment, the plug computer also includes a memory card adapter to operate as a content cache or, more generally, a local content storage device for patient-specific content.

In one embodiment, the content storage burden associated with individual patient network nodes 690 is distributed across several patient network nodes such that the content delivered to a particular patient may be supplied via a respective patient network nodes or via a nearby patient network nodes.

In one embodiment, the system comprises two main components; namely, a small server for administrative/content delivery and a number of content presentation devices or players. That is, the system 600 of FIG. 6 is modified to retain only the following server and player components (as well as the network connecting them to each other). Specifically, any location where audio/audiovisual presentation is desired will have a player (e.g., each patient's room, one or more common areas, etc.). The server may be located in a communications closet for the facility so that it can have easy access to the internal wireless network as well as the internet. The server houses the entire music collection while each player holds the media it specifically requires. The server keeps track of each player and sends updates one at a time on an as needed basis. The audio files are kept in a compressed format balancing fidelity with data size. Altogether, these strategies are adapted to avoid network over utilization.

Media programs held on the server, such as podcasts, may be updated from the remote/Internet content sources on a periodic basis. These updates are optionally scheduled at night to eliminate internet congestion with normal business activity. Podcasts may be vocal programs, rather than music, and use lower bit rate compression as higher quality audio is not required.

Non-audio data kept on the server may be limited to patients' name, room number, and time schedules for the audio/audiovisual programs. Each player may hold only its respective time schedule.

For music and system maintenance purposes, external access to the server is a provided to administrative personnel and/or 3rd parties servicing the system. This can be in the form of a virtual private network (VPN), Remote Desktop access or other mechanism. To improve security, the server and players can be separated from other network devices by using a VLAN or other common network strategies.

In various embodiments, the ADL comprises four main programs; namely, WAKE, ENERGIZE, RELAX and SLEEP. These main programs operate as boundaries in terms of the type of content that may be scheduled for a particular patient as well as the type of content that may be requested by a patient on-demand. The ADL is also modified to accomplish various goals of the facility, such as calming a patient down prior to a move or visit. Generally speaking, while content for a patient is selected in accordance with patient tastes and interests, only content conforming to the content prescription associated with the patient and conforming to the ADL will be presented to the patient.

The above-described physical and logical mechanisms provide a system for providing appropriate content/music therapy prescriptions, including content storage, content delivery and content presentation mechanisms. As will be appreciated by those skilled in the art informed by the teachings of the present disclosure, various modifications may be made with respect to these physical and logical mechanisms without departing from the systems, methods and apparatus contemplated by inventors. Several particular embodiments utilizing the teachings of the various figures will now be discussed in more detail.

ElderCare and Other Facility Types

The various embodiments discussed herein provide a therapeutic audio/audiovisual enrichment service having-utility within the context of treating patients at eldercare facilities, hospitals, prisons, schools and other types of institutions which benefit from the calming, motivating, therapeutic and/or other effects provided by music or content therapy. Music, music therapy, spirituality, educational pieces, current events and audio books may all be individually tailored and delivered directly to the resident's room. Schedules are set up in advance so no staff intervention is required, and in the event of an unscheduled request, staff members can accommodate with just a few mouse clicks.

As a participant, each resident or patient receives a Music or Content Prescription based on medical condition, acuity level, personal preferences and interests. For music and music therapy, careful consideration is also given to arrangement, tempo, genre, key, volume and desired outcome. Group participation may be encouraged. Groups can cooperatively listen to audio books, lectures and current events while improving socialization and assisting in cognitive stimulation.

Software Access

The software/firmware used within the context of various embodiments provide two levels of access: facility and administrative. A facility level of access offers all the necessary functions for day-to-day use. These functions may include:
Log In
Find a Patient
Quick Play
Adjust Volume
View/Modify a Patient Schedule
Add Notes/Send Comments

An administrative level of access offers the above-described functions for day-to-day use, as well as the following additional rights and responsibilities, including:
Add a Patient
Edit a Patient
User Profile Management

Content Therapy is More than Music

Music therapy is the primary content therapy discussed above with respect to the various embodiments. However, what are you visual content such as movies, television shows and special-purpose audiovisual presentations (e.g., particular combinations of color, light, movement and/or sound) are also appropriate for use within the context of the various embodiments.

Group Therapy

In one embodiment, where the content/music therapy appropriate to one patient is appropriate to multiple patients, these multiple patients are scheduled to receive simultaneous presentation of the content/music therapy. In one variation, the simultaneous presentation of such content/music therapy is provided a common room such that the patients experience a sense of community with respect to be presented content/music.

Delivery of Spiritual Support

Presently, spiritual support given to patients of institutions mainly comprises visits to the institutions by local religious leaders. It is believed that patients benefit greatly when their spiritual needs are addressed. Thus, various embodiments discussed herein are modified to define and provide content intended to address the spiritual needs of the patients within, illustratively, an institution. These embodiments help hospitals and other extended care institutions or facilities meet their patients' needs.

In one embodiment, the content delivered to patients is intended to address their spiritual needs. Specifically, various embodiments provide spiritual support to patients by providing content of a spiritual or religious nature. Such spiritual/religious content may be provided via podcast, streaming media, file transfer or any other technique to an institutions server and/or individual presentation device.

Spiritual/religious content may comprise religious or, more generally, spiritual services associated with the denomination of a patient, such as Christianity, Judaism, Islam or any other major religion, minor religion or spiritual philosophy. Spiritual/religious services may be provided in accordance with the ADL, the denomination of the patient, the type for purpose of the spiritual/religious service and/or other factors. Spiritual/religious services may comprise those services normally provided according to a calendar associated with a particular denomination, specific services provided by spiritual/religious leaders on behalf of the patient, or any other type of spiritual/religious content appropriate to the patient in terms of taste, denomination, ADL and/or prescription.

In one embodiment, patients sharing a common faith or denomination gather at the predefined location to receive spiritual/religious services together as a community. In other embodiments, patients received spiritual/religious services individually, such as where such patients cannot be safely moved.

On-Demand Delivery of Content

In one embodiment, a patient may elect to receive specific content/music for presentation rather than no content, default content and/or previously scheduled content. In this embodiment, a patient utilizes remote control device 698 to “order” specific content via interacting with a user interface supported by the presentation device 696. In one embodiment, the patient may select for on-demand presentation any available content. In other embodiments, the patient may only select for on-demand presentation only that content conforming to the ADL. Specifically, the patient may request content that is within the subset of content appropriate to the particular time of day (e.g., morning wake-up, afternoon relaxation and the like), the particular goals of the institution of facility (e.g., preparing for a patient move, preparing for administration of a new drug, waiting for a doctor or family visit and the like), and/or content of a specific type (e.g., music, audiovisual, voice messages, text messages and the like).

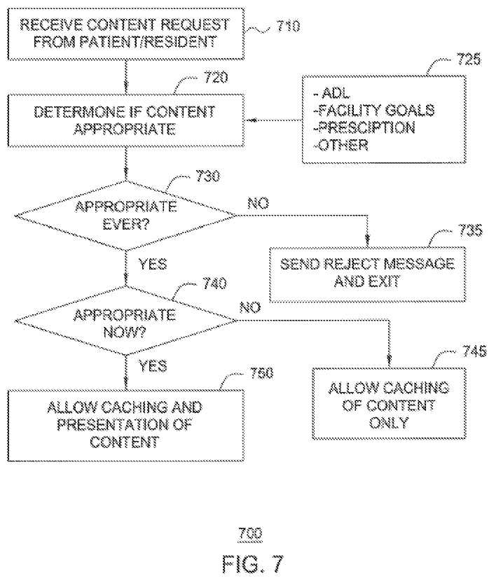

FIG. 7 depicts a flow diagram of a method according to one embodiment. Specifically, the method 700 of FIG. 7 is entered at step 710, when the server receives a content request from a patient. At step 720 a determination is made as to whether the requested content is appropriate for the patient. Referring to box 725, the appropriateness of the requested content is determined with respect to one or more of the ADL, the facility goals, the content prescription of the patient and or other criteria.

At step 730, if the requested content is not ever appropriate, then the method 700 proceeds to step 735 where a rejection message is sent to the patient in the method exits.

At step 740, if the requested content is appropriate but not appropriate at this time, then the method 700 proceeds to step 745 where the requested content is allowed to be cached by the patient, but not allowed to be presented to the patient.

At step 750, if the requested content is appropriate at this time, then the requested content is allowed to be cached by the patient and allowed to be presented to the patient.

Message Content Distribution

One embodiment of the invention is adapted to disseminating audio, video and/or text messages to patients. Specific, in this embodiment of the invention, the family, friends, doctors and so on associated with the patient may transmit messages to the patient using audio, video and/or text media or content. These messages may be delivered to the facility for subsequent transmission using e-mail, direct connection (e.g., via a browser interface with the facility website), a telephone call and the like. These messages may be therapeutic in nature or merely informative in nature.

In this embodiment, messages will be provided to the patient in conformance with the content/music prescription requirement as well as the ADL. It is likely to be the case that message content cannot be provided on an immediate basis. In this case, the message content will be stored at the facility server or patient network node and presented in conformance with the next opportunity is indicated by, illustratively, the ADL.

In one embodiment, the transmitter of message content to a patient may indicate the type of message content, such as “emergency” content, “non-emergency” content were some other type of message content.

Message content may be provided to patients as it is an opportunity exists as defined by the ADL and relevant prescriptions, or a set time each day. In one embodiment, messages are provided to patients during state transitions only.

Various embodiments described above provide a system of assessing patient affinities for therapeutic music, assessing specific music adapted to those affinities and efficiently providing individualized patient therapeutic music in accordance with patient vital signs, patient daily activity requirements, institutional governance and/or control requirements, caregiver requests and so on.

Various embodiments operate to provide most or all of the benefits of individualized therapeutic music and media within the context of a institutional environment as one example. Various embodiments provide initial scheduling of musical therapy based upon patient affinity and institutional scheduling. In certain embodiments, scheduled therapeutic music delivery is adapted in response to changes in institutional goals, patient preferences, caregiver requests, patient requests and/or patient vital signs. For example, in response to particular events such as security breaches, patient deaths and the like, individualized music therapies adapted to calm all patients may be employed irrespective of scheduled therapeutic music delivery.

Various embodiments described above include embodiments such as those listed below in the enumerated clauses; namely:

1. A method for delivering therapeutic content to patients, comprising: defining for each patient a respective playlist including prescribed content conforming to patient tastes; defining for each patient respective activities of daily living (ADL) schedules including at least one time period for receiving therapeutic content; and providing therapeutic content to each patient according to the patient's respective playlist and ADL.

2. The method of clause 1, further comprising adapting the provided therapeutic content in response to changes in patient vital signs.

3. The method of clause 2, wherein the changes in patient vital signs are indicative of the patient achieving a desired state in conformance with the ADL.

4. The method of clause 3, wherein the desired state comprises any of a wake state, an energized state, a relaxed state and a sleeping state.

5. The method of clause 1, further comprising adapting the provided therapeutic content in response to changes in one or more of institutional goals, patient preferences, caregiver requests, patient requests and patient vital signs.

6. The method of clause 1, wherein the therapeutic content comprises music.

7. The method of clause 1, wherein the therapeutic content comprises audiovisual content.

8. The method of clause 1, wherein the therapeutic content comprises message content.

9. The method of clause 8, wherein the message content comprises one or more of audio, video and text content.

10. The method of clause 9, wherein message content is only provided during changes state transitions according to the ADL.

11. The method of clause 1, further comprising adapting the provided therapeutic content in response to a patient on-demand content request where the requested content conforms to the ADL.

12. The method of clause 1, wherein the same therapeutic content is provided to each member of a group of patients having a common ADL portion.

13. The method of clause 1, wherein the same therapeutic content comprises any of a music, audiovisual content and message content.

14. The method of clause 13, wherein the same therapeutic content supports a common therapy need or spiritual need of the group members.

15. A system for delivering therapeutic content to patients, comprising a playlist generator, for processing health information and content preference information associated with a patient to generate a respective content prescription playlist; a scheduler, for storing an activity of daily living (ADL) schedule for the patient, the ADL schedule including at least one time period for receiving therapeutic content; and a media server, for propagating prescribed content to a patient according to the playlist and ADL schedule associated with the patient.

16. The system of clause 15, wherein the system performs the steps of playlist generation, ADL scheduling and prescribed music propagation for each of a plurality of patients within an institution.

17. The system of clause 15, wherein playlist generator, scheduler and media server are implemented using administrative equipment within a facility, the system further comprising a network, for communicating therapeutic content from the administrative equipment to respective patient network nodes.

18. The system of clause 15, wherein each patient network node is associated with a respective patient and operative to communicate therapeutic content toward a presentation device associated with the respective patient.

19. The system of clause 18, wherein each patient network node further includes a storage device to store therapeutic content prior to communicating therapeutic content toward the storage device.

20. The system of clause 17, wherein the network comprises a wired network.

21. The system of clause 17, wherein the network comprises a wireless network.

22. The system of clause 17, wherein each of the patient network nodes comprises a remote control device supporting patient interaction with the administrative equipment.

23. The system of clause 15, wherein the scheduler responsively adapts the therapeutic content provided to a patient in response to changes in patient vital signs.

24. The system of clause 23, wherein the changes in patient vital signs are indicative of the patient achieving a desired state in conformance with the ADL, the desired state comprises any of a wake state, an energized state, a relaxed state and a sleeping state.

25. The system of clause 15, wherein the scheduler responsively adapts the therapeutic content provided to a patient in response to changes in one or more of institutional goals, patient preferences, caregiver requests, patient requests and patient vital signs.

26. The system of clause 15, wherein the therapeutic content comprises music, audiovisual content or message content.

27. A computer program product wherein computer instructions, when processed by a computer, adapt the operation of the computer to perform a method for delivering therapeutic content to patients, the method comprising: defining for each patient a respective playlist including prescribed content conforming to patient tastes; defining for each patient respective activities of daily living (ADL) schedules including at least one time period for receiving therapeutic content; and providing therapeutic content to each patient according to the patient's respective playlist and ADL.

Improvements to the above-described embodiments, as well as new embodiments, tools, processing modules and the like are also provided and described herein.

### Content Description/Characterization Module & Prescriptive Playlist Generation Module

Various embodiments are directed to a content description/characterization module configured for automatic processing of content/media (e.g., music, poetry, prayer, etc.) to objectively characterize the content/media (or portions thereof) in accordance with a descriptive system developed by the inventors, wherein a prescriptive playlist generator retrieves characterized content (or portions thereof) for use in generating a prescriptive playlist configured to effect a therapeutic result when presented to a patient/user (e.g., audio or audiovisual presentation).

Various embodiments utilize a deep learning system to determine additional patterns and characteristics to further refine models for Feature Slopes and the like configured to achieve therapeutic purposes.

Various embodiments utilize fewer (e.g., 9) or more (e.g., 50 or more) characteristics/musical dimensions or subsets thereof, to describe content/media (e.g., music, poetry, prayer, etc.), wherein differing sub-sets of these characteristics/musical dimensions are more appropriate to use for differing outcomes. For example, fewer characteristics/musical dimensions may provide sufficient characterization for Energy programs, whereas adding 12 more particular characteristics/musical dimensions may be appropriate for Relax programs.

Various embodiments utilize Valence as a key feature—i.e., a measure of the “positiveness” of a song (happy, sad, angry, relaxed).

Additional figures, presentation, discussion and the like are provided herein and associated with numerous embodiments of the invention, which embodiments may be implemented in conjunction with the above-described embodiments, as independent embodiments, or in any combination thereof. Various below embodiments comprise computer-implemented systems, modules, mechanisms or portions thereof such as using special purpose or general purpose computing devices including processing, memory, and input/output functions. Various embodiments may be implemented using one or more dedicated computer servers, clusters of servers, an Infrastructure as a Service (IaaS) system, communications/interfacing mechanisms and the like, such as a computing environment providing memory and compute resources configured to instantiate virtual machines or containers configured to host software components such as microservices, applications, control modules and the like in accordance with the various functional elements described herein.

### Overview

Various embodiments comprise systems, methods, architectures, mechanisms and apparatus for generating an audio segment playlist configured to provoke a physiological response in a listener in accordance with a desired outcome category, comprising: selecting, from a features database, a plurality of audio segments having features associated with both listener information and the desired outcome category; and ordering within the playlist at least a portion of the selected audio segments in accordance with at least one feature progression associated with the outcome category. In response to a change in desired playlist runtime, or a change in listener information, further selected audio segments may be added to the playlist or existing audio segments may be deleted from the playlist. The feature progression may be formed using one or more of the following tempo, brightness (timbre), brightness variability, pulse strength, pulse variability, proportion in major key(s), tonal clarity, tonal clarity variability, tonal entropy, or other objectively determinable features. The feature progression may be formed using one or more of acousticness, danceability, energy, intrumentalness, loudness, speechiness, tempo and valence, or other subjective features compound features (i.e., a combination of progressions such as slopes or shapes defining existing features over the playlist). The desired outcome category of the playlist may comprise an energy category, and the feature progression comprises a positive Tempo Feature Slope (TFS). The desired outcome category of the playlist may comprise a relax category, and the feature progression comprises a negative Tempo Feature Slope (TFS).

In some embodiments, prior to inclusion in the feature database, each audio segment is processed using at least two feature extraction tools configured to extract therefrom respective sets of audio segment features. In some embodiments, responsive to common audio segment features extracted by different feature extraction tools being different, an average of the common audio segment features will be included in the feature database. In some embodiments, responsive to a statistical distance between common audio segment features extracted by different feature extraction tools being indicative of a feature extraction error, the feature data indicated as erroneous will not be included in the feature database.

### Personalized Therapeutic Music Playlist Construction

Various embodiments provide a system, apparatus, and method for construction of a personalized therapeutic music playlist. Specifically, various embodiments enable the creation of a custom music playlist (e.g., a compilation of songs/sounds in a specific order) configured to provide for a listener/patient a desired physiological or mental outcome at that moment in time or a future moment in time. This music playlist is the Music Prescription™.

Various embodiments contemplate a computer implemented method to generate a music playlist based on a user's current emotional and physiological state, desired purpose, and music genre. The music playlist is a collection of songs/sounds in a specific order and is based on personal preferences, music interests, potential medical data, and/or other factors as discussed herein. In operation, a user selects (or is assigned by caregiver or pre-scheduled by a caregiver) a desired music genre, such as 1990s Country or Classic Rock & Roll, and a purpose. The purpose could be a physiological outcome such as increase energy or encourage relaxation, or the purpose could be for an activity such as dining. A processor takes this input and generates a music playlist comprised of an ordered list of original music recordings using machine learning methods.

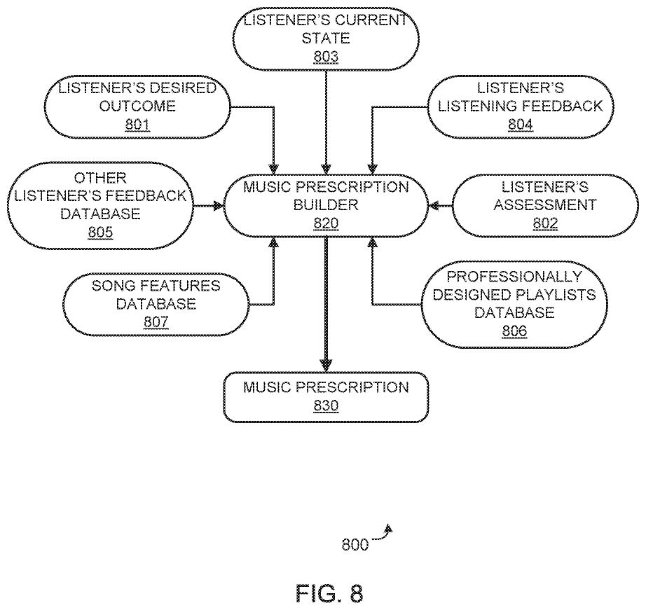

FIG. 8 graphically depicts a process flow in accordance with various embodiments. Specifically, the process flow 800 of FIG. 8 contemplates that a music prescription builder (MPB) 820 implemented via a computing device receives takes multiple inputs into consideration to determine the appropriate content to be consumed by the listener.

As depicted in FIG. 8, the inputs to the MPB 820 may comprise one or more of the following:

Desired outcome for the listener 801—the desired change in physiological and mental state that the generated music playlist should encourage and support, such as outcomes to encourage/support includes states such as relaxation, energy, dining (digestion), etc.

Listener assessment 802—history, background, issues, diagnoses. The relevant data may come from the listener, a medical professional, a professional music therapist, electronic medical records, and/or other data gathering tools/mechanisms. The history and background may include music likes and dislikes, region where grew up, life history such as veteran, religious faith, education level, work history, age, gender, etc. Issues and diagnoses may include physiological and mental items such as cardiac issues, depression, Alzheimer's and the like (e.g., if someone has cardiac issues then a selection of songs with less musical complexity in instrumentation, vocalization or tempo may be appropriate).

Listener's current state 803—mood, live monitored vital signs. For example, mood may be defined using the Abraham-Hicks Emotional Guidance Scale. Vital signs can be heart rate, respiration rate, body or skin temperature; measurements related to sleep quality, attention level, concentration level; facial expressions, tapping of hands or feet, changes in eye contact, and the like.

Database of listener's previous listening feedback 804—likes and dislikes for specific songs and/or other relevant information.

Database of other listeners' feedback 805—assessments, states, and previous listening feedback 805—what was the impact (objective or subjective) to other listeners.

Database of professional music designer generated playlists 806—playlists are specific to desired outcomes within music genres.

Database of song features 807—analysis data from public data source, industry standard analysis tools, and a song analyzer such as described below with respect to the various embodiments. In particular, song (audio segment/file) features such as beat per minute, tempo, key, instrumental-ness, energy level, lyric sentiment analysis, and the like may provide static or time varying representations of the song (audio segment/file) sufficient to enable characterization and subsequent use of the song (audio segment/file) as part of a therapeutic playlist. Relevant tools and data sources may include Librosa (librosa.org) audio and music processor, Spotify's published analysis, LyricFind, sentiment analysis such as via Google's natural language processing and the like.

Broadly speaking, the MPB 820 algorithm takes all received input information and applies machine learning methods to generate a custom music playlist for the listener. Listener feedback may be collected during and after the music playlist, such as the collecting/storing of feedback for use in generating future playlists. Also collected/stored may be listeners' vital signs, indicators of mood, contemporaneous indications of positive or negative feedback and so on.

Various embodiments contemplate implementing the MPB within the context of a broader application wherein application providers may use their own music, streaming accounts, or existing playlist building processes in conjunctions with an MPB layer configured to put third party application songs in a correct order for a desired outcome.

Various embodiments contemplate the addition of visual components to a Music Prescription to further enhance effectiveness. The visual components may comprise still or moving imagery which may be selected based on an individual's personal preferences, historical data, collaborative filtering, and known research on imagery with certain types of music. Various embodiments contemplate allowing individuals (or businesses using MPB-based products) to choose from their own library of static or video imagery.

### Music Prescription Process

Within the context of a music prescription process as contemplated herein, three main components are of interest; namely, a song analyzer, a music prescription builder (MPB), and a user deliver mechanism.

The song analyzer is used to characterize music, which may be broadly defined as any sound, sequence of sounds, and/or portion thereof. The term song as used herein is intended to broadly denote any of a musical song, a voice presentation such as a lecture or sermon, a natural sound recording, an artificially generated sound recording, and/or any other audible information. The song analyzer may use artificial intelligence (AI), machine learning (ML), and/or other techniques to break down “songs” into constituent feature vectors or other representations so as to assign the songs to specific charactered clusters based on dimensionality components, composite scores (based on key musical characteristics), and/or other factors.

The MPB creates therapeutic music programs by arranging specific songs (audio segments or portions thereof) in a specific order based to achieve a desired outcome for a particular patient/listener.

The MPB process may be configured and updated in response to general research of individual patient data pertaining to the physiological impact of music to the brain, the application of music therapy best practices, the use of key elements in songs to create programs/prescriptions designed to evoke a desired outcome in a listener/patient, and (optionally) the use of listener/patient-relevant faith-based content to further the desired outcome.

The user deliver mechanism enables a listener/patient to self-select forma list of programs based on desired outcome combined with the genre, style, and/or other preferences of the listener/patient.

### Song Features

The song analyzer is used to characterize each song in accordance with various features extracted via analysis of the song or portions thereof to create a database of song features. The song features are descriptive of the song or portion(s) thereof. Song components or features of interest my include musical genre, vocalization, instrumentation, timbral brightness, clarity, tempo (e.g., beats per minute), time spent in major keys, texture, pulse strength, lyrical sentiment, and so on. There are may different features that may be used to analyze and characterize songs (i.e., audio information).

Some features characterize a song or portion thereof, and do not change for the duration of the song or portion thereof. For example, a song may be associated with a particular genre (classic rock, techno, gospel, etc.).

Some features change during the presentation of a song or portion thereof. For example, changes in tempo (increase or decrease in the pace or speed of music such as measured by beats per minute) may occur frequently during a song or portion thereof.

Various embodiments utilize slope (change over time) of specific features/components of interest (i.e., “feature slopes”) of individual songs or portions thereof to create a playlist comprising songs and/or song portions which, when presented to a patient/listener, evokes a desired physiological response in that patient/listener (e.g., wake up, fall asleep, feel more energetic, feel more relaxed, etc.). That is, multiple songs (any type of audio selections) or portions thereof may be arranged to provide a playlist exhibiting at the playlist level one or more desired features or feature slopes.

Various embodiments analyze each song using multiple analysis tools or sources, such as analysis data from public data source, industry standard analysis tools, third party tools, proprietary song analyzers and the like. Relevant tools and data sources may include Librosa (librosa.org) audio and music processor, Spotify's published analysis, LyricFind, sentiment analysis such as via Google's natural language processing and the like. Where conflicting output data such as different feature information about a song is provided by multiple tools, various embodiments provide a data quality control (DQC) processing function/module to resolve the differences and provide correspondingly useful feature data to the database. Standard statistical processing methods may be used for the DQC function, such as statistical differencing to identify the potential scope of a conflict, deletion of clearly or likely bad data, averaging of seemingly reasonable but conflicting data, and so on.

In particular, song (audio segment/file) features such as beat per minute, tempo, key, instrumental-ness, energy level, lyric sentiment analysis, and the like may provide static or time varying representations of the song (audio segment/file) sufficient to enable characterization and subsequent use of the song (audio segment/file) as part of a therapeutic playlist.

### Playlists

Unique “Playlists” are defined as existing within a combination of Purpose, Genre, Style, and Program. Some, but not all such groupings contain multiple playlists (or sets of songs). Also, the same song can occur in multiple playlists. Crucially, Playlists are not necessarily conceived as some collection of songs that share some set of attributes/features but in which the ordering of songs does not matter, but rather as a specific ordering of songs with some progression of characteristics/features/attributes in mind. Thus, to the extent that the progressing characteristics correspond to one or more quantifiable features/properties of the music, such trends should (a) be discernible within the existing playlists, and (b) serve as the basis for a classification engine that would be capable of playlist construction in terms of ordering of songs.

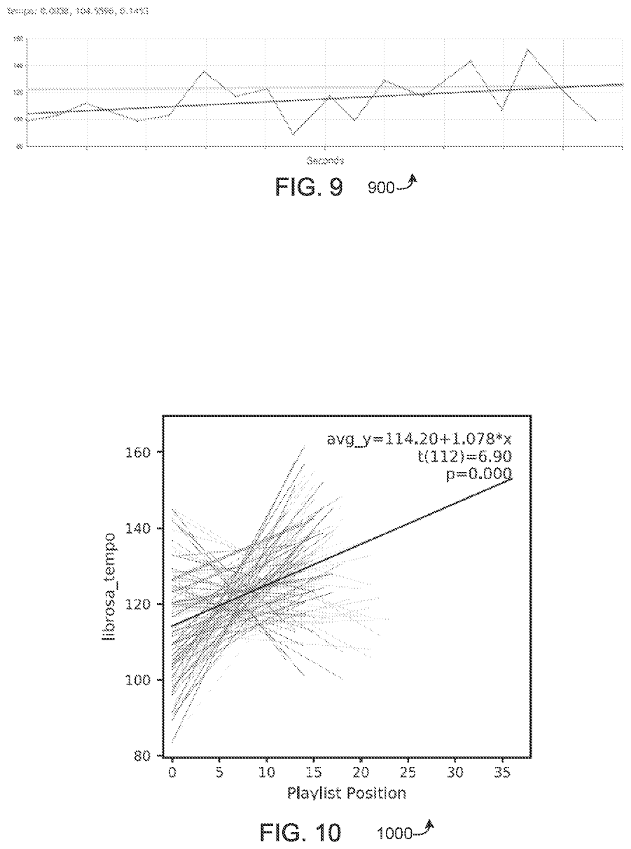

FIG. 9 graphically depicts a Tempo Feature Slope (TFS) of an exemplary music prescription playlist generated in accordance with an embodiment. Specifically, FIG. 9 depicts a feature progression of a playlist comprising TFS progressing upwards throughout a music program, such as an “Energy Program” where such an increase in tempo is configured to evoke an increase in subjective energy or activity for the patient/listener. It can be seen by inspection that the tempo of each song is plotted as a function of time or location within the playlist, and that the average tempo of the playlist increases over time.

Other examples of playlist generation are associated with features (e.g., the nine features discussed herein), feature levels, slopes, changes associated with feature slopes, and so on. Changes in features may include increases or decreases in a slope of a curve fitted to a representation of the feature displayed as a function of time (or playlist location). For example, features of songs (audio segments or portions thereof) that may be used alone or in combination to provide a relevant feature progression for use in defining a playlist may comprise one or more of these or other features: tempo, brightness (timbre), brightness variability, pulse strength, pulse variability, proportion in major key(s), tonal clarity, tonal clarity variability, tonal entropy, acousticness, danceability, energy, intrumentalness, loudness, speechiness, tempo and valence, and other features or compound/combination features.

FIG. 10 graphically depicts a Tempo Feature Slope (TFS) for each of a plurality of energy playlists, along with an average of the TFSs of the plurality of playlists. It can be seen by inspection that the general trend in terms of TFS for each of the energy playlists is positive (i.e., increasing tempo over time).

Other statistical representations may also be used with respect to characterizing changes in features over time or playlist location, such as changes in spectral centroid which are indicative of changes in a center of mass of a spectral representation of a feature slope.

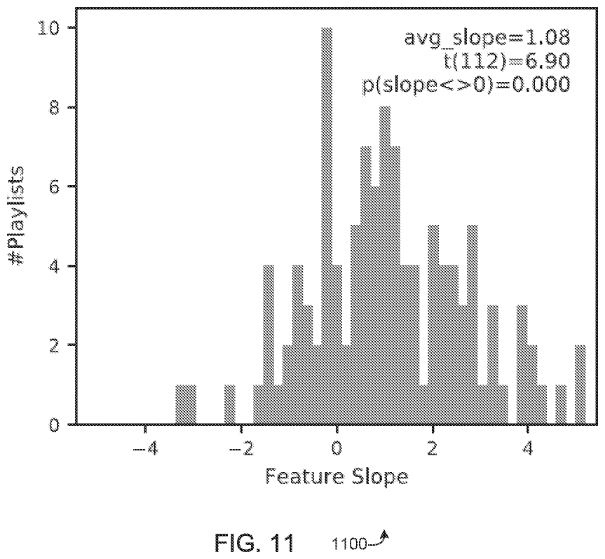

FIG. 11 is a histographic representation of the distribution of tempo feature slopes for the energy playlist of FIG. 10. It can be seen by inspection that the spectral representation of the time variant data of FIG. 10 is associated with a particular shape in the histographic representation of that data in FIG. 11, and which may be characterized as having a center of mass or spectral centroid. That is, systematic variation across songs for playlists in each of the various categories may be further described.

Examples of categories to which playlists may be generated include various categories of desired patient/listener outcome, including:
“Energy” where over time the songs presented via the playlist exhibit the characteristics of tempo increases (positive tempo feature slope), spectral centroid increases, spectral centroid variability decreases, and proportion of a song in a major key decreases.
“Relax” where over time the songs presented via the playlist exhibit the characteristics of tempo decreases (negative tempo feature slope), the degree to which the music is clearly within a key (tonal clarity) increases, the number of different keys or amount of harmonic movement within the piece (tonal entropy) decreases.
“Wake” where over time the songs presented via the playlist exhibit the characteristics of tempo increases (positive tempo feature slope), spectral centroid decreases, the amount of variability in the beat (pulse variability) increases, the number of different keys or amount of harmonic movement within a piece (tonal entropy) decreases.
“Sleep” where over time the songs presented via the playlist exhibit the characteristics of tempo decreases (negative tempo feature slope), spectral centroid decreases, spectral centroid variability decreases, proportion of song in a major key increases, the degree to which the music is clearly within a key (tonal clarity) increases, variability in the degree to which the music is clearly within a key (tonal clarity variability) decreases, the number of different keys or amount of harmonic movement within a piece (tonal entropy) decreases.

The inventors have determined that while characterizing songs (audio segments) or portions thereof such as via 9-dimensional feature vector analysis is useful in identifying which of those songs or portions thereof will be of interest to a particular patient/listener, a more important function is to determine the position of such songs within a playlist are optimal to evoke the desired physiological response of the patient/listener. For example, referring to the “energy” playlist described above, given a desire to increase song tempo over a song playlist, lower tempo songs of interest are placed toward the beginning of the playlist while higher tempo songs are place toward the end of the playlist. Similarly, given a number of previously generated “energy” playlists, the spectral centroid associated with a playlist being generated may be allowed to increase though the variability of the spectral centroid should be constrained or decrease. Finally, the proportions of a currently presented song in major key may be allowed to decrease.

A further option with respect to playlists is shortening or expanding the runtime of the playlist in response to available time and/or other factors. Within the context of the various embodiments, the removal of songs or portions thereof from a playlist should be performed in a manner that does not alter the features, feature slopes, or other criteria associated with the playlist so that the playlist still functions as a mechanism to evoke a desired physiological response in the patient/listener for the relevant category of responses (i.e., energy, relax, wake, sleep, etc.).

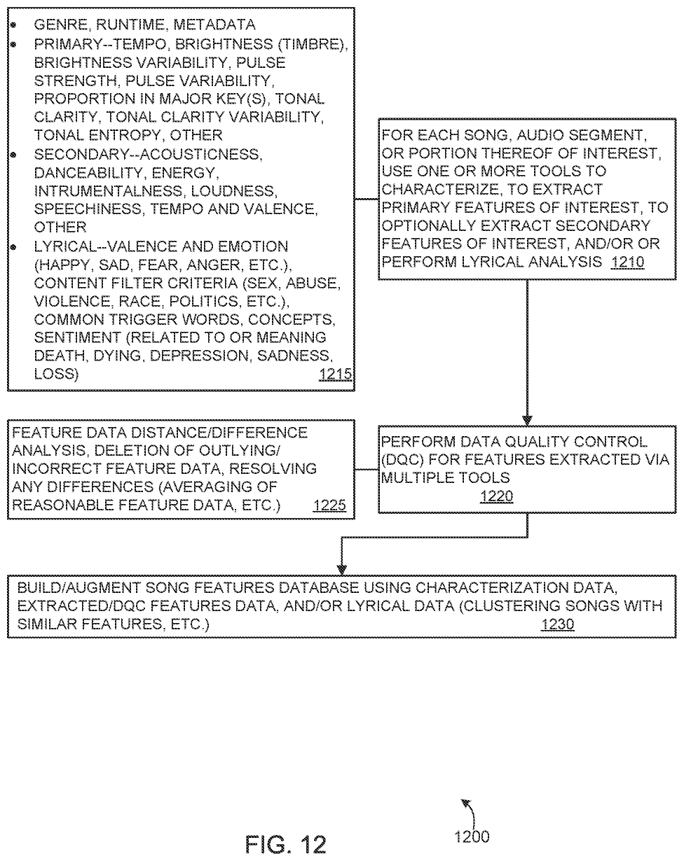

FIG. 12 depicts a flow diagram of a features database update method according to an embodiment. Specifically, the steps of FIG. 12 are discussed above and herein.

At step 1210, for each song (audio segment, or portion thereof) of interest, one or more tools are used to characterize the song, to extract primary features of interest therefrom, to optionally extract secondary features of interest therefrom, to optionally perform lyrical analysis of the song, and so on. Referring to box 1215, such characterizations may include song genre, runtime, metadata and the like. Primary features of interest may comprise tempo, brightness (timbre), brightness variability, pulse strength, pulse variability, proportion in major key(s), tonal clarity, tonal clarity variability, tonal entropy, and other objectively determinable features. Secondary features of interest may include acousticness, danceability, energy, intrumentalness, loudness, speechiness, tempo and valence, and other subjective features or compound features. Lyrical analysis may comprise natural language processing of the song to characterize tone or content, such as valence and emotion (e.g., happy, sad, fear, anger, etc.), common content filter criteria (e.g., sex, abuse, violence, race, politics, etc.), common trigger words, concepts, or sentiment (e.g., related to or meaning death, dying, depression, sadness, loss, etc.), and so on.

At step 1220, in the case of conflicting output data such as different feature information about a song being provided by multiple tools, various embodiments provide a data quality control (DQC) processing function/module to resolve the differences and provide correspondingly useful feature data to the database. Standard statistical processing methods may be used for the DQC function, such as statistical differencing to identify the potential scope of a conflict, deletion of clearly or likely bad data, averaging of seemingly reasonable but conflicting data, and so on.

At step 1230, the song-related data from steps 1210 and 1220 is used to build or augment a song features database

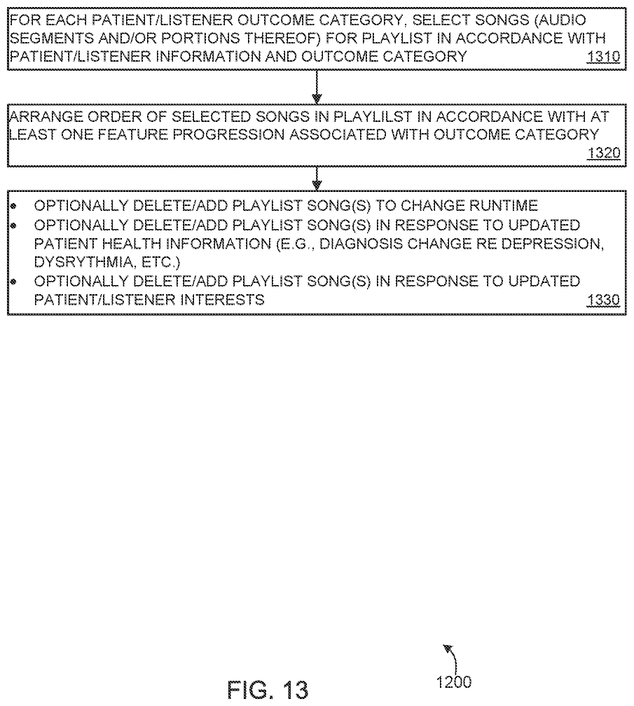

FIG. 13 depicts a flow diagram of a playlist generation method according to an embodiment. Specifically, the steps of FIG. 13 are discussed above and herein.

At step 1310, for each patient/listener outcome category, a plurality of songs (audio segments and/or portions thereof) are selected for possible inclusion in a playlist in accordance with patient/listener information, outcome category, and the like such as discussed above.

At step 1320, the selected songs (audio segments and/or portions thereof) are arranged in an order or sequence within the playlist in accordance with at least one feature progression association with the outcome category, such as discussed above.

At step 1330, various options may be invoked as needed to adjust the playlist contents. In some embodiments, one or more songs (audio segments and/or portions thereof) are optionally deleted from or added to the playlist, preferably in a manner that retains the playlist feature slope or other characterizing indicia associated with the desired outcome category. Such options may be in response to various situations, such as:
in response to a newly defined time period for a patient prior to sleep or meal time, to change (increase or decrease) the runtime of the playlist;
in response to updated patient health information indicative of a the patient being better served by avoiding songs with triggering words in case of depression, avoiding booming high tempo songs in case of cardiac dysrhythmia, avoiding songs with excessive speechiness in case of stroke victims, etc.;
in response to updates/changes in the patient/listener interests (e.g., different genre, etc.)

Various elements or portions thereof depicted and described herein with respect to FIGS. 8-13 provide functions implemented at least in part via computing devices, data storage devices, and the like. These elements or portions thereof contemplate the use of computing devices of various types, though generally a processor element (e.g., a central processing unit (CPU) or other suitable processor(s)), a memory (e.g., random access memory (RAM), read only memory (ROM), and the like), various communications, input/output and the like.

As such, the various functions depicted and described herein may be implemented at the elements or portions thereof as hardware or a combination of software and hardware, such as by using a general purpose computer, one or more application specific integrated circuits (ASIC), or any other hardware equivalents or combinations thereof. In various embodiments, computer instructions associated with a function of an element or portion thereof are loaded into a respective memory and executed by a respective processor to implement the respective functions as discussed herein. Thus various functions, elements and/or modules described herein, or portions thereof, may be implemented as a computer program product wherein computer instructions, when processed by a computing device, adapt the operation of the computing device such that the methods or techniques described herein are invoked or otherwise provided. Instructions for invoking the inventive methods may be stored in tangible and non-transitory computer readable medium such as fixed or removable media or memory, or stored within a memory within a computing device operating according to the instructions.

Various embodiments a music prescription algorithm implemented via one or more computing devices utilizes machine learning methods to create music playlists specific to a single listener. The machine learning methods may analyze known good data to discover patterns that can be applied to create new data with similar characteristics/features. Specific to the embodiments, the steps are divided into a training and discovery phase, and the playlist creation phase.

With respect to the training and discovery phase (such as described herein with respect to FIG. 12), existing tools (such as Librosa) and data sources (such as Spotify) are used to create a database of characteristics/features for every song in the collection. These characteristics/features could include, but are not limited to, tempo, average audio frequency, music key, pulse strength, etc. These characteristics/features are stored in a database for easy retrieval later. A trained Music Designer may have created multiple music playlists for a specific purpose and music genre. This is the collection of known good data. These machine learning methods may be sued to analyze these playlists to discover the trajectories of various characteristics/features of each song within the playlist. These trajectories are stored in a database for easy retrieval later. Additional data may be collected from published therapeutic music studies regarding the physiological effects on a listener when songs with certain characteristics/features are heard. For example, a listener with cardiac issues should listen to songs with less complexity, lower tempo, etc. This physiological effects data is stored in a database for each retrieval later. These training and discovery steps are repeated as songs are added to the collection, as music designers create new music playlists, and as new related studies are discovered.

With respect to the playlist creation phase (such as described herein with respect to FIG. 13), a listener selects a music genre and purpose. The machine learning methods retrieve the previously stored trajectories for the selected music genre and purpose. The machine learning methods retrieve the collection of characteristics/features of the songs for the selected music genre. The machine learning methods retrieve the physiological effects data. The machine learning methods retrieve the physiological data of the listener. The machine learning methods select a subset of songs in the selected music genre and assemble them in an order such that the trajectories of the songs' characteristics/features in the new playlist closely match the training data. The song selection process compares the physiological condition of the listener with the physiological effects data when deciding to include the song in the subset. The created playlist is delivered to the listener.

A machine learning method used in various embodiments is a linear regression. The analysis of the known good data (playlists) calculates the starting value and slope of each song characteristic over time. The songs in the created playlist are ordered such that the trajectory in the multi-dimensional space of all characteristics/features is closely similar to the known good playlists. More advanced methods may be applied, specifically a non-linear analysis, but will always follow a similar training-creation process.

Thus, in various embodiments, a features database and feature progressions relevant to outcome categories are generated using a machine learning tool operative to process audio segments known to be relevant to the respective outcome categories.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like. It will be appreciated that the term “or” as used herein refers to a non-exclusive “or,” unless otherwise indicated (e.g., use of “or else” or “or in the alternative”).

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

What is claimed is:

1. A computer-implemented method of generating an audio segment playlist configured to provoke a physiological response in a listener in accordance with a desired outcome category, the method comprising:
selecting, from a features database, a plurality of audio segments having features associated with both listener information and the desired outcome category; and
ordering within the playlist at least a portion of the selected audio segments in accordance with at least one feature progression associated with the outcome category.

2. The method of claim 1, further comprising adding selected audio segments to the playlist or deleting audio segments from the playlist in response to a change in desired playlist runtime.

3. The method of claim 1, further comprising adding selected audio segments to the playlist or deleting audio segments from the playlist in response to updated listener information.

4. The method of claim 1, wherein the feature progression is formed using at least one of tempo, brightness (timbre), brightness variability, pulse strength, pulse variability, proportion in major key(s), tonal clarity, tonal clarity variability, tonal entropy, and other objectively determinable features. Secondary features of interest may include acousticness, danceability, energy, intrumentalness, loudness, speechiness, tempo and valence, and other subjective features or compound features.

5. The method of claim 1, wherein the desired outcome category of the playlist comprises an energy category, and the feature progression comprises a positive Tempo Feature Slope (TFS).

6. The method of claim 1, wherein the desired outcome category of the playlist comprises a relax category, and the feature progression comprises a negative Tempo Feature Slope (TFS).

7. The method of claim 1, wherein prior to inclusion in the feature database, each audio segment is processed using at least two feature extraction tools configured to extract therefrom respective sets of audio segment features.

8. The method of claim 7, further comprising:
responsive to common audio segment features extracted by different feature extraction tools being different, determining that an average of the common audio segment features will be included in the feature database.

9. The method of claim 7, further comprising:
responsive to a statistical distance between common audio segment features extracted by different feature extraction tools being indicative of a feature extraction error, determining that the corresponding feature will not be included in the feature database.

10. The method of claim 1, wherein the features database and feature progressions are generated using a machine learning tool operative to process audio segments relevant to the outcome category.
